(19) United States
(12) Patent Application Publication
(10) Pub. No.: US 2007/0105817 A1
(43) Pub. Date: May 10, 2007

(54) Use of cicletanine and other furopyridines for treatment of systolic-predominant hypertension, isolated systolic hypertension, elevated pulse pressure, and general hypertension

(76) Inventors: Jim Page, Laramie, WY (US); Karen Page, Laramie, WY (US); Glenn V. Cornett, Palo Alto, CA (US)

Correspondence Address: GREENBERG TRAURIG, LLP (SV2), 2450 Colorado Avenue, Suite 400E, Santa Monica, CA, 90404, US

(21) Appl. No.: 11/356,158

(22) Filed: Feb. 15, 2006

Related U.S. Application Data
(60) Provisional application No. 60/735,632, filed on Nov. 9, 2005.
(60) Provisional application No. 60/758,524, filed on Jan. 11, 2006.

(51) Int. Cl. A61K 31/724; A61K 31/4741

(52) U.S. Cl. 514/58; 514/302

(57) ABSTRACT

This invention provides therapeutic compositions of cicletanine and other furopyridines for the treatment of, elevated pulse pressure or isolated systolic hypertension, as well as general hypertension, in monotherapy and in combined therapy with other anti-hypertensive agents (such as organic and inorganic nitrogen donors, calcium channel blockers, diuretics, beta blockers, angiotensin receptor blockers, ACE inhibitors, aldosterone antagonists, renin inhibitors and centrally-acting antihypertensives) cardiovascular agents (such as medications to treat heart failure) and oral antidiabetic agents (such as biguanides and glitazones). Such compositions include enantiomerically pure (positive or negative) embodiments, as well as enantiomeric mixtures other than a racemic mixture, and include daily dosages of less than 50 mg. Further provided are methods of treatment of general or systolic hypertension, wherein patients are administered the inventive compositions, either a monotherapeutic furopyridine composition, or a combination therapy, which includes a second agent in addition to the furopyridine, for treatment of general hypertension or systolic hypertension, and hypertension-associated complications.

### RELATED APPLICATIONS

The present application claims priority to U.S. Provisional Patent Application Ser. No. 60/735,632 of Page et al., filed on Nov. 9, 2005, and to U.S. Provisional Patent Application Ser. No. 60/758524 of Page et al., filed on Jan. 11, 2006, both applications entitled “The use of cicletanine and other furopyridines for the treatment of elevated pulse pressure and isolated systolic hypertension”. The present application further claims priority to U.S. patent application Ser. No. 11/232,724, of Cornett and Page, filed on Sep. 21, 2005, entitled “Enantiomer compositions of cicletanine, alone and in combination with other agents, for the treatment of diseases”.

### FIELD OF THE INVENTION

The field of this invention relates to the use of racemic and non-racemic furopyridine compounds and derivatives thereof, alone and in combination with other therapeutic agents, for prophylaxis and/or treatment of systolic predominant hypertension, isolated systolic hypertension, elevated pulse pressure, and general hypertension.

### BACKGROUND

Pulse pressure is the difference between systolic pressure and diastolic pressure; elevated pulse pressure is commonly the result of a disproportionately high systolic pressure. It is now believed that elevated systolic pressure, or the consequently elevated pulse pressure, is more strongly associated with cardiovascular adverse events than diastolic blood pressure (The Seventh Report of the Joint National Committee on Prevention, Detection and treatment of high Blood Pressure, NIH Publication No. 03-5233, May 2003). Current anti-hypertensive therapies are not fully satisfactory for the generally older population that suffers from this “isolated systolic hypertension”. The current standard of practice therapies tend either to be insufficiently effective at reducing blood pressure in a broad sense, or they tend to produce a lowering of both systolic and diastolic pressures, which leaves the pulse pressure relatively uneffected. Further, the therapies that do show some ability to exert a differential effect, do not provide an adequate total reduction in systolic blood pressure.

Highlighting the desirability of being able to therapeutically achieve a differential effect, or systolic-preferential effect, is recent evidence that excessive reduction in diastolic pressure may be associated with increased adverse cardiovascular and cerebrovascular adverse events. Another difficulty with some current hypertension therapies is that their efficacy does not persist over time. This loss of effectiveness is generally attributed either to short serum half life, or to rapid development of tachyphylaxis, i.e., a loss in effectiveness of a drug after an initial use, or under continuous use, that is ascribed to receptor desensitization or down regulation.

Current hypertension therapy, and therapies for isolated systolic hypertension are incompletely satisfactory for other reasons as well. The problem presented by endothelial dysfunction, in particular the deficit of vascular nitric oxide (NO) remains a challenging factor in the pathology of hypertension, and currently available drugs for treatment of hypertension do not include within the scope of their mechanisms of action an effective approach to treating endothelial dysfunction. New or improved forms of therapy that are directed preferentially toward reducing systolic blood pressure and pulse pressure, and which resolve any of the therapeutic deficiencies just described, would be a welcome addition to the healthcare market.

### SUMMARY OF THE INVENTION

This invention and its embodiments relate to the therapeutic use of racemic and non-racemic cicletanine, other furopyridines, salts thereof, esters thereof (including salts with H₂O coordinated bonds), other noncovalent derivatives, such as complexes, clathrates, and chelates thereof, as well as metabolic products derived from administered furopyridines which may, themselves, provide therapeutic benefit. These furopyridine-based compounds are used alone, in various combinations within the furopyridine genus, as well as collectively as a genus in combination with other agents, for treatment of systolic predominant hypertension, isolated systolic hypertension, elevated pulse pressure, and general hypertension.

Embodiments of the invention include cicletanine treatment, at low-nanomolar concentrations, that are sufficient to correct deficits in vascular nitric oxide, and to do so safely and effectively, both as a furopyridine-based monotherapy, and in combination with other hypertension agents. More particularly, some embodiments of the invention further provide for the use of cicletanine and other furopyridines for the treatment of human systolic predominant hypertension, isolated systolic hypertension, elevated pulse pressure, and general hypertension in monotherapy and in combination with organic and inorganic nitrogen donors, nitric oxide synthase modulators, antioxidants, calcium channel blockers, beta blockers, angiotensin receptor blockers, ACE inhibitors, aldosterone antagonists, renin inhibitors, centrally-acting antihypertensives, diuretics, and other compounds used to treat hypertension, cardiovascular diseases (such as heart failure and angina), or metabolic disease.

Embodiments of the invention include a therapeutic formulation comprising a furopyridine composition, such composition referring to any formulation comprising one or more furopyridine compounds, including derivatives and metabolites of such furopyridine compounds as they may arise in the body of a patient upon treatment with the furopyridine composition, as described above, or any combination thereof. By way of example, one such furopyridine species is cicletanine : (+/−)-3-(4-chlorophenyl)-1,3-dihydro-6-methylfuro-[3,4-c]pyridin-7-ol); another species is (+/−) 3-(4-fluorophenyl)-1,3-dihydro-7-hydroxy-6-methylfurop-[3,4-c] pyridine. In various embodiments, each of the one or more furopyridine compounds may be present in various enantiomeric proportions or profiles, including the substantially pure positive (+) enantiomer, substantially pure negative (−) enantiomer, a racemic mixture of the (+) and (−) enantiomers, and a non-racemic mixture of the (+) and (−) enantiomers. Such non-racemic mixtures may be weighted toward either the (+) and (−) enantiomer, varying, for example from a (+) to (−) ratio from between an extreme of about 99 to 1 to the other extreme of 1 to 99.

Embodiments of the invention include total furopyridine daily dosages of less than 50 mg, wherein the total dosage accounts for the combined dosage of positive (+) and negative (−) enantiomers of the furopyridine species. Cicletanine, for example, may be administered to a subject in amounts of about 25 mg, about 30 mg, about 35 mg, about 40 mg, or about 45 mg. Other embodiments of the invention include compositions that comprise more than one furopyridine species; in such cases, embodiments further include those in which the daily dosage of the combined concentrations of the separate furopyridine species is less than the cicletanine-bioequivalent of 50 mg, accounting for differences in molecular weight, potency, and bioavailability.

Further embodiments of the invention include combination therapies, wherein the above described furopyridine compositions, at above identified daily dosages of less than 50 mg, are combined with other so-called second agents, such agents themselves being effective for the treatment of hypertension. Such second agents may include, for example, organic and inorganic nitrates, calcium channel blockers, and diuretics.

Typically, embodiments of the invention are used as therapeutic formulations for human patients or subjects; these formulations may be appropriate for treatment of ongoing disease of any stage of progression or severity, as well as prophylaxis for patients medically considered to be at risk for the development of hypertensive disease. Embodiments of the invention are also applicable to the veterinary uses. Typical embodiments of the formulation of the invention are for oral use; other embodiments are for administration by any conventional mode of administration, including injection, intravenous administration, and any form of parenteral administration. Embodiments of formulation of the invention may include non-medicinal constituents (i.e., non-furopyridine and non-second therapeutic agent) that help the effectiveness or bioavailability of the biologically active agents. Such additives to the formulation may include absorption enhancers, tissue selectivity enhancers, tissue adhesion enhancers, polymers, and other agents to improve stability and bioavailability, half-life in vivo, duration of effect, and/or effectiveness of drug delivery to appropriate target tissues.

Embodiments of the invention also include methods of treatment, in which patients suffering from elevated pulse pressure, systolic predominant hypertension, or isolated systolic hypertension are treated by the administration of the therapeutic compositions described herein. In some cases, patients with borderline systolic hypertension, or with generalized hypertension, or patients who are considered to be at risk of progressing toward isolated systolic hypertension may benefit from treatment with these furopyridine compositions, and accordingly, embodiments of the invention include methods for treating these patients as well.

Various embodiments of the inventive treatment, in addition to the administration of furopyridine compositions at dosages described, may further include the generation of metabolites of the administered furopyridines in the body of the patient, where such metabolites, themselves, may be responsible for- or contribute to medically beneficial effects. Formulations and methods of treatment with such formulations, as provided by embodiments of the present invention, are understood to be therapeutically sufficient or effective when the treatment results in a clinically apparent improvement in any clinical sign or symptom associated with any of the aforementioned forms of hypertension, as measured or assessed by a responsible health care professional, working in the bounds of currently accepted standards of practice, or as perceived by a cognizant and reasonable patient being provided the inventive treatment.

### DESCRIPTION OF EMBODIMENTS OF THE INVENTION

Furopyridine Treatment Preferentially Directed Toward Systolic Hypertension

In one embodiment of the invention, cicletanine (chemical name: ±-3-(4-chlorophenyl)-1,3-dihydro-6-methylfuro-[3,4-c]pyridin-7-ol) or other furopyridine compounds are used (1) at doses resulting in serum blood level measurements below those usually associated with substantial diuretic and direct vasodilatory effects, or (2) at doses near or below the currently-marketed minimum daily dosage of 50 mg. These compounds, at these dosages, can exert differential effects on systolic and diastolic hypertension, with an effect on lowering systolic pressure and, in contrast, either a much smaller effect-, or an absence of a detectable effect on diastolic hypertension. In the cases of both monotherapy and combination therapy (i.e., in combination with a second agent, as described below), per embodiments of the invention, the furopyridine composition may also have one or more the following effects: exert protective effects on the vasculature, and/or reduce or slow the development of dementia, a common side effect of hypertension, as well as end-organ pathologies associated with diabetes.

The differential therapeutic effects on hypertension, biased toward alleviating the systolic pressure and reversing endothelial dysfunction, may be applicable in patients with higher levels of systolic hypertension. For example, the effects are persistent over both the short term and the longer term: observable at time intervals 24 hours or more after dosing, developing over a period of a few weeks, and continuing at 12 weeks of continued daily dosing. It is believed that the effects are derived from mechanisms of action different from those of other currently available compounds that are applied to the treatment of systolic hypertension and elevated pulse pressure, and complementary to the effects of such compounds. In particular, cicletanine and other furopyridine compounds contribute to reducing the development of tachyphylaxis to organic and inorganic nitrogen donors.

The differential effects, or systolic-preferential effects, of embodiments of the invention are obtained at relatively low drug levels in blood, levels that minimize or avoid invoking other active mechanisms common to cicletanine and other furopyridine compounds, as well as minimizing the likelihood of eliciting tachphylaxis. In some patients, however, for example those with a mix of both systolic and diastolic hypertensive characteristics, higher dosing may be needed to adequately address the reducing of systolic and diastolic pressures, and pulse pressure, without unduly lowering diastolic pressures. Carefully tailored dosing will allow adjustment of the individual effects of the cicletanine or other furopyridines, particularly (in the case of cicletanine) at daily dosages of less than 50 mg, and in combination with other therapeutic compounds (see “second agent” description, below) to obtain one or more benefits such as reduced tachyphylaxis, extended duration of action, improved side effect profile, improved convenience, enhanced organ protective effects, and modulation of systolic blood pressure, diastolic blood pressure, and pulse pressure.

Daily Dosage Ranges of Furopyridine Compositions

Embodiments of the invention include total furopyridine dosages of less than 50 mg per day, wherein the total dosage accounts for the combined dosage of positive (+) and negative (−) enantiomers of the furopyridine species. Cicletanine, for example, may be administered to a subject in amounts of about 25 mg, about 30 mg, about 35 mg, about 37.5 mg, about 40 mg, about 42.5 mg, about 45 mg, or about 47.5 mg. Some embodiments may include dosages either lower or higher than this particular range, depending on medical features particular to the patient, such as age, weight, sex, affliction with conditions or complications other than hypertension, and/or interactions with other drugs. Daily dosages lower than 25 mg, may include, for example dosages stepping up from 1 μg, to 3 μg, to 10 μg, to 30 μg, to 100 μg, to 300 μg, to 1 mg, to 3 mg, to 10 mg, to 24 mg. Specific examples of lower daily dosages include about 5 mg, about 7.5 mg, about 10 mg, about 12.5 mg, about 15 mg, about 17.5 mg, about 20 mg, and about 22.5 mg. Higher daily dosing may be needed for some patient populations due to impaired absorption or enhanced metabolism and excretion.

In some embodiments, the differential anti-hypertensive effect is obtained with conventional oral dosing formulations, but other embodiments include formulations that provide reduced first-pass metabolism and/or more constant serum levels may provide more consistent effects. Such formulations include those designed, merely by way of example, to be depot injectable or implantable; formulations may be particularly adapted for transdermal, transmucosal, oral delayed release, oral delayed gastric discharge, or any other facilitator of pharmacokinetic effectiveness as known by practitioners of the art. Such and similar technologies may provide more predictable and consistent serum levels that are adequate to provide the desired differential anti-hypertensive effect, but not so high as to diminish the differential anti-hypertensive effect by being beyond the level appropriate to the medical requirements of the individual patient.

In some embodiments, this differential anti-hypertensive effect is applicable and beneficial due to the high incidence of isolated systolic hypertension and elevated pulse pressures in the hypertensive population in general. Older hypertensive patients are particularly at risk, as isolated systolic hypertension accounts for 54% of hypertensive patients of 50 to 59 years of age, and 87% of patients of 60 or more years of age. Further, it is believed that elevated pulse pressure is more closely associated with adverse cardiovascular events than is diastolic blood pressure.

Furopyridine Compositions Varying with Respect to Relative Enantiomeric Presence

Embodiments of the invention include compositions of cicletanine or other furopyridines that vary in terms of the relative presence of positive (+) and (−) enantiomers (see below). These varied compositions may be used as a monotherapy or in combination therapy, with second agents, to treat human elevated pulse pressure, systolic predominant hypertenison, isolated systolic hypertension, or general hypertension. In general terms, these compositions varying with respect to their enantiomeric profile, can take the following forms:
1. Pure (+) cicletanine or other furopyridine enantiomer,
2. Non-racemic compositions of cicletanine (NRC) or other furopyridines, involving a mixture of (+) cicletanine or other furopyridine and (−) cicletanine or other furopyridine where the ratio of (+) to (−) is greater than 1:1,
3. Racemic cicletanine: a mixture of (+) cicletanine or other furopyridine and (−) cicletanine or other furopyridine where the ratio of (+) to (−) is 1:1,
4. Non-racemic cicletanine (NRC) or other furopyridine involving a mixture of (+) cicletanine or other furopyridine and (−) cicletanine or other furopyridine where the ratio of (+) to (−) is less than 1:1, and
5. Pure (−) cicletanine or other furopyridine enantiomer.

By enantiomeric compositions being “pure” is meant “substantially pure”, i.e., pure by standard methods of analysis, including the respective margin of error in the method. In the case of non-racemic mixtures, in various embodiments, where the ratio of (+) to (−) is lesser or greater than 1:1, a wide range in relative presence is meant, for example, a range in ratios varying from one extreme of between about 1% (+) ::99% (−) to the other extreme of about 99% (+) ::1% (−). More particularly, the ratio of (−) enantiomer:: (+) enantiomer may, for example, be about 95::5, about 90::10, about 80::20, about 70::30, about 60::40, about 55::45, about 40::60, about 30::70, about 20::80, about 10::90, or about 5::95. Other embodiments may include variations of these ratios, occupying the approximate midpoint range thereof.

Embodiments of the invention include treatment with furopyridines other than cicletanine. An example of such is (+/-) 3-(4-fluorophenyl)-1,3-dihydro-7-hydroxy-6-methylfurop-[3,4-c] pyridine. This compound can be produced in a racemic mixture and can be used in either purified enantiomer condition or in a weighted, non-racemic enantiomeric mixture. Other furopyridine compounds have been identified, by Garay, et al., for example, (“Stimulation of K+ fluxes by diuretic drugs in human red cells”; Biochemical Pharmacology 33, #13, 2013-2020, 1984).

Those of ordinary skill in the art will recognize furopyridines as a genus, and that other furopyridine compounds or species exist, and that other novel compounds may be synthesized in the future; all such furopyridines and their derivatives, as described above, are included as embodiments in the present invention. Embodiments of the invention further include compositions that include more than one furopyridine, each present at total dosage levels independent of the other within the constraints of total daily dosing as described herein, and each of which could be present, respectively, as one of the five enantiomeric profiles described above. Compositional embodiments of medical treatment provided by the invention further include metabolites of furopyridines that are made within the body following administration of the furopyridines at the dosage levels described herein, even metabolites that are not currently known. And method of treatment embodiments of the invention include receiving the medically beneficial effects of such metabolites.

Furopyridine Compositions Combined with Second Agents for Treatment of Hypertension

Embodiments of the present invention include combination therapies, wherein a furopyridine composition is combined with a second agent. “Second agent”, as used herein, refers to any therapeutic agent other than the furopyridine compositions. “Second agent” is also general term that may refer to a plurality of non-furopyridine agents, in that a combination therapy could include more than one second agent. Such second agents may be, by themselves, effective agents for lowering blood pressure, and/or treating any complications associated high blood pressure. Second agents may also include agents not established as conventional agents for treatment of hypertension, particularly at dosages in which they are combined with the furopyridine compositions, but which, in combination with furopyridine compositions at the dosages described herein, the combination becomes an effective therapeutic formulation. As described herein, the furopyridine compositions used in combination with second agents are administered at daily dosages described above, and in all the enantiomeric variations described above.

Particular embodiments of the invention thus include a cicletanine composition (in its various enantiomeric forms) in combination with one or more second agents. Such agents include, by way of example, organic and inorganic nitrates, calcium channel blockers, diuretics, angiotensin converting enzyme inhibitors, angiotensin receptor blockers, beta blockers, centrally-acting antihypertensives, aldosterone antagonists, renin inhibitors, endothelin receptor antagonists, other antihypertensives, and other agents used to treat disease states associated with endothelial dysfunction, including, by way of example, oral antidiabetic agents, lipid-lowering agents, and agents that increase high density lipoprotein (HDL) cholesterol levels. One still more particular embodiment of the invention is that of furopyridine compositions in combination with calcium-channel blockers or nitrates (organic or inorganic), as these are currently preferred treatments for isolated systolic hypertension. The mechanisms of furopyridine action and those of these second agents are different, and therefore complementary of each other; their combined broadly-defined antihypertensive effect may be additive or synergistic.

In some embodiments, as with the monotherapy description above (i.e., monotherapeutic in that the furopyridine composition is not combined with a non-furopyridine second agent), patients with borderline systolic hypertension, or with generalized hypertension, or patients who are considered to be at risk of progressing toward isolated systolic hypertension may benefit from treatment with these combination therapies, and accordingly, embodiments of the invention include methods for treating these patients as well.

### EQUIVALENTS OF THE INVENTION

While particular embodiments of the invention and variations thereof have been described in detail, other modifications and methods of using the disclosed therapeutic combinations will be apparent to those of skill in the art. Accordingly, it should be understood that various applications, modifications, and substitutions may be made of equivalents without departing from the spirit of the invention or the scope of the claims. Various terms have been used in the description to convey an understanding of the invention; it will be understood that the meaning of these various terms extends to common linguistic or grammatical variations or forms thereof. It will also be understood that when therapeutic agents have been identified by trade names or common names, that these names are provided as contemporary examples, and the invention is not limited by such literal scope, particularly when agents have been further described in terms of chemical class and/or by mechanism of action. Although the description offers biochemical theory and interpretation, it should be understood that such theory and interpretation do not bind or limit the claims. Further, it should be understood that the invention is not limited to the embodiments that have been set forth for purposes of exemplification, but is to be defined only by a fair reading of the appended claims, including the full range of equivalency to which each element thereof is entitled.

We claim:

1. A formulation comprising a furopyridine composition, the composition comprising one or more furopyridine compounds, wherein the daily dosage of the composition is less than 50 mg of cicletanine bioequivalent, the formulation sufficient for the treatment of any of systolic-predominant hypertension, isolated systolic hypertension, elevated pulse pressure, and general hypertension.

2. The formulation of claim 1 wherein the furopyridine composition comprises one or more furopyridine derivatives, the derivatives selected from the group consisting of salts, salts with H₂O coordinated bonds, esters, clathrates, and chelates.

3. The formulation of claim 1 wherein the formulation is for oral use.

4. The formulation of claim 1 wherein the daily dosage is between about 1 μg and about 47.5 mg.

5. The formulation of claim 1 wherein the daily dosage is between about 1 mg and about 45 mg.

6. The formulation of claim 1 wherein the daily dosage is selected from the group consisting of about 5 mg, about 7.5 mg, about 10 mg, about 12.5 mg, about 15 mg, about 20 mg, about 25 mg, about 30 mg, about 35 mg, about 37.5 mg, about 40 mg, and about 45 mg.

7. The formulation of claim 1 wherein the furopyridine composition comprises cicletanine.

8. The formulation of claim 1 wherein the enantiomeric profile of each of the one or more furopyridine compounds of the composition is selected from the group consisting of substantially pure positive (+) enantiomer, substantially pure negative (−) enantiomer, a racemic mixture of the (+) and (−) enantiomers, and a non-racemic mixture of the (+) and (−) enantiomers.

9. The formulation of claim 1 further comprising at least one second therapeutic agent to form a combination therapy.

10. The formulation of claim 9, wherein the second agent is selected from the group consisting of organic and inorganic nitrogen donors, nitric oxide synthase modulators, antioxidants, calcium channel blockers, beta blockers, angiotensin receptor blockers, ACE inhibitors, aldosterone antagonists, renin inhibitors, centrally-acting antihypertensives, and diuretics.

11. The formulation of claim 1 further comprising one or more bioavailability enhancers selected from the group consisting of absorption enhancers, tissue selectivity enhancers, tissue adhesion enhancers, and polymers.

12. A method of treating a patient with any one or more of systolic-predominant hypertension, isolated systolic hypertension, and elevated pulse pressure, the method comprising administering the patient a formulation comprising a furopyridine composition, wherein the daily dosage of the composition is less than 50 mg cicletanine bioequivalent.

13. The method of claim 12, wherein the formulation further comprises a second therapeutic agent.

14. The method of claim 12, wherein the treating of the patient further comprises deriving therapeutic benefit from any metabolite derived from the furopyridine composition in the body of the patient.

15. A method of treating a patient with generalized hypertension by administering the patient a formulation comprising a furopyridine composition comprising one or more furopyridine compounds, wherein the daily dosage of the composition is less than 50 mg cicletanine equivalent.

16. The method of claim 15, wherein the formulation further comprises a second therapeutic agent.

17. The method of claim 15, wherein the treating of the patient further comprises deriving therapeutic benefit from any metabolite derived from the furopyridine composition in the body of the patient.
